(19) United States
(12) Patent Application Publication
(10) Pub. No.: US 2003/0028084 A1
(43) Pub. Date: Feb. 6, 2003

(54) Remote psychoanalysis system using operation history

(76) Inventor: Tadashi Kaneko, Narashino-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/119,157

(22) Filed: Apr. 10, 2002

(30) Foreign Application Priority Data
Aug. 1, 2001 (JP) 2001-233511

(51) Int. Cl. A61B 5/00

(52) U.S. Cl. 600/300

(57) ABSTRACT

A remote psychoanalysis system using operation history that allows any person to be subjected to psychological counseling, being not restricted by time and distance. The remote psychoanalysis system using operation history comprises the steps of a client 2 preparing psychoanalysis object information by using a client computer apparatus 1, and transmitting the psychoanalysis object information to a server computer apparatus 10 through an internet 30; the server performing accounting for the client through said communication means for each matter of psychoanalysis request or confirmation of reception of money for the accounting; the server 11 transmitting said psychoanalysis object information prepared by the client from the server computer apparatus 10 to a plurality of analyzer computer apparatuses 20 through the internet 30; and some analyzer 21 of a plurality of analyzers 21 practicing psychoanalysis of the pertinent client 2 on the basis of said psychoanalysis object information acquired by using his analyzer computer apparatus 20, and transmitting the psychoanalysis object information to the client computer apparatus 1 through the internet 30.

### BACKGROUND OF THE INVENTION

1. Field of the Invention

The present invention relates to an innovative remote psychoanalysis system using operation history that has not existed up to now.

2. Prior Art

In the modern society, where the community, the home, the school, the company and any other things surrounding a person can cause stresses, it is desirable that persons be subjected to this type of counseling in good time, regardless of the age and the gender, for making social livelihood healthily.

However, to be subjected to such type of counseling, the patient must generally attend to the psychiatry of a specific hospital, a metal hospital, the office of a clinic psychologist, or the like, and when a person actually attends to such a hospital or office, there is the possibility of that the acquaintances, the relatives and other surrounding persons turn a strange look upon him, thus persons tend to hesitate to be subjected to counseling.

In addition, when a person is engaged in business or study, for example, he cannot easily be subjected to such a type of counseling because of the scantiness of time and the distance to the location.

### SUMMARY OF THE INVENTION

The present invention has been developed in consideration of the above conventional situation, and the purpose of it is to provide a remote psychoanalysis system using operation history that allows any person to be subjected to psychological counseling easily and rapidly, being not restricted by time and distance.

To achieve this purpose, the invention as stated in claim 1 provides a remote psychoanalysis system using operation history having a client computer apparatus on the side of a client requesting psychoanalysis; a server computer apparatus on the side of a server controlling psychoanalysis information; a plurality of analyzer computer apparatuses on the side of analyzer practicing psychoanalysis; and communication means connecting said client computer apparatus, said server computer apparatus, and said plurality of analyzer computer apparatuses to one another through a network, comprising the steps of: the client preparing psychoanalysis object information by using the client computer apparatus, and transmitting the psychoanalysis object information to the server computer apparatus through the communication means; the server performing accounting for the client through said communication means for each matter of psychoanalysis request or confirmation of reception of money for the accounting; the server transmitting said psychoanalysis object information prepared by the client from the server computer apparatus to the plurality of analyzer computer apparatuses through the communication means; and some analyzer of the plurality of analyzers practicing psychoanalysis of the pertinent client on the basis of said psychoanalysis object information acquired by using his analyzer computer apparatus, and transmitting the psychoanalysis object information to the client computer apparatus through said communication means.

The invention as stated in claim 2 provides a remote psychoanalysis system using operation history as claimed in claim 1, wherein said psychoanalysis object information and said psychoanalysis result information are transmitted/received as a ciphered electronic mail.

The invention as stated in claim 2 provides a remote psychoanalysis system using operation history as claimed in claim 1 or 2, wherein, when said analyzer prepares analysis result information stating that the metal state of the client is unsteady, the analysis result information is transmitted to the client computer apparatus as an immediate-opening-disabled electronic mail, which cannot be opened before a specified date.

According to the present invention, a series of psychoanalysis processes are easily and rapidly performed through communication means, thus, psychoanalysis in a remote place (when the analyzer and the client are distant from each other) becomes possible, and preparing the psychoanalysis object information, making the analysis, and receiving the analysis result can be performed, both the analyzer and the client being liberated from the restriction by distance and time.

Further, according to the present invention, the procedure which has been taken for preparing the psychoanalysis object information can be reproduced in either the forward or backward direction; reversible psychological counseling and investigation can be performed; large-scale psychological counseling and analysis can be easily performed; and quantitative data of psychological counseling can be stored.

Further, according to the present invention, the psychoanalysis object information and the psychoanalysis result information are transmitted/received as an enciphered electronic mail, thus if the information should leaked to some other computer apparatus connected to the communication means, the privacy of the client and the analyzer can be protected by the encryption provided, and by sending the psychoanalysis result information as an immediate-opening-disabled mail, the metal pain of the client can be alleviated.

### BRIEF DESCRIPTION OF THE DRAWINGS

FIG. 1 is a block diagram showing the system configuration of a remote psychoanalysis system of an embodiment of the present invention;

FIG. 2 is an explanatory drawing showing the process of psychoanalysis object information being prepared by a client;

FIG. 3 is an explanatory drawing showing the process of the psychoanalysis object information being transmitted by the client;

FIG. 4 is an explanatory drawing showing the process of the psychoanalysis object information being transmitted from the server to the analyzer;

FIG. 5 is an explanatory drawing showing the process of the psychoanalysis object information being analyzed by the analyzer;

FIG. 6 is an explanatory drawing showing the process of the psychoanalysis result information being transmitted to the client; and

FIG. 7 is an explanatory drawing showing the process of the psychoanalysis result information being received and stored by the client.

### DESCRIPTION OF THE PREFERRED EMBODIMENTS

Hereinbelow, an embodiment of the present invention will be described in details with reference to the attached drawings.

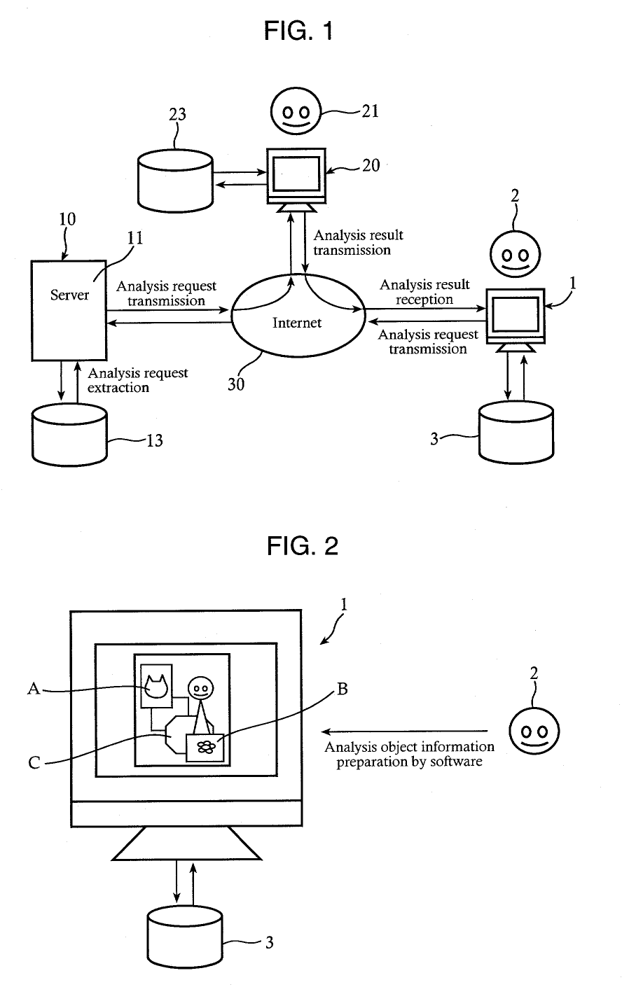

FIG. 1 is a block diagram showing the entire configuration of a remote psychoanalysis system using operation history of the present embodiment, and this remote psychoanalysis system using operation history comprises a client computer apparatus 1 on the side of a client 2 requesting psychoanalysis; a server computer apparatus 10 on the side of a server 11 controlling psychoanalysis information; a plurality of analyzer computer apparatuses 20 (only one is shown in FIG. 1) on the side of an analyzer 21; and an internet 30, which is communication means connecting said client computer apparatus 1, said server computer apparatus 10, and said plurality of analyzer computer apparatuses 20 to one another through a network.

The client computer apparatus 1 has a client database 3. The server computer apparatus 10 has a server database 13. The plurality of analyzer computer apparatuses 20 have an analyzer database 23.

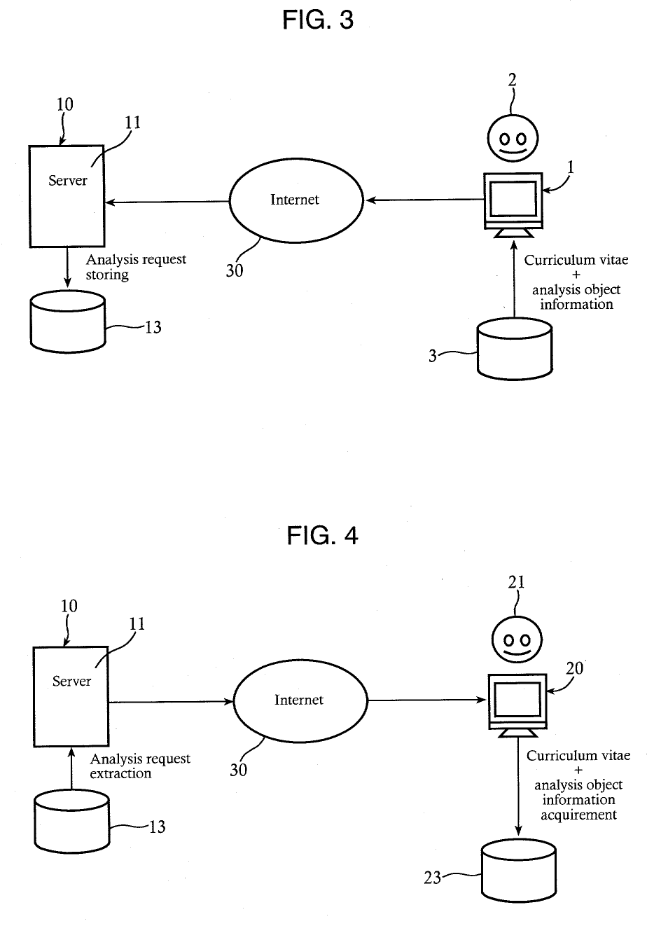

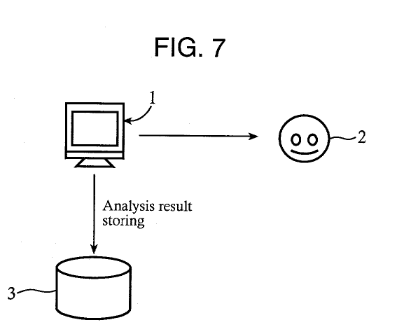

Next, a series of processes of psychoanalysis of the client 2 performed by use of the remote psychoanalysis system as configured above will be explained with reference also to FIG. 2 to FIG. 7.

First, the client 2 prepares psychoanalysis object information by himself with the use of the client computer apparatus 1, and transferred this psychoanalysis object information as a file appended to the electronic mail from the client computer apparatus 1 to the server computer apparatus 10 through the internet 30. In this case, the psychoanalysis object information is ciphered by means of encryption software previously loaded in the client computer apparatus 1 before being transferred.

As shown in FIG. 2, the psychoanalysis object information is prepared by the client 2 operating the client computer apparatus 1 by software, e.g., disposing a photo A on an optional coordinate point [20,30] on the screen of the client computer apparatus 1; disposing a photo B on an optional coordinate point [100,300] on the screen of the client computer apparatus 1; and drawing a circle C having a radius length [30] on the point [100,300] (as in the collage therapy), with a curriculum vitae of the client 2 being attached to it.

The psychoanalysis object information prepared is transferred through the internet 30, being also stored in the client database 3.

The server 11 stores the psychoanalysis object information including said curriculum vitae in the server database 13 by means of the server computer apparatus 10, and after the server 11 performing accounting for the client 2 through the internet 30 for each matter or manual or automatic confirmation of the reception of the money for the accounting on the basis of the psychoanalysis object information about the client 2 that is stored in said server database 30, the server 11 transfers said psychoanalysis object information prepared by the client to the plurality of analyzer computer apparatus 20 from the server computer apparatus 10 through the internet 30 as a file appended to the electronic mail by specifying a particular analyzer 21 or without specifying a particular analyzer 21.

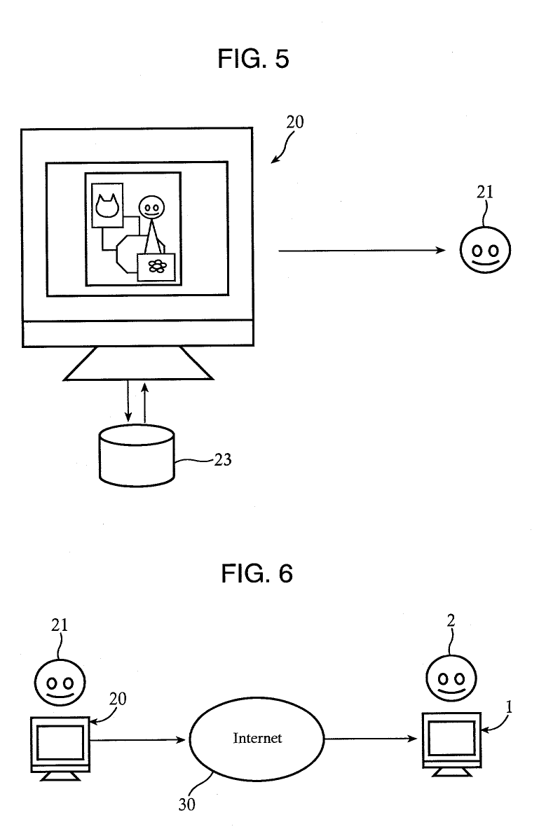

As shown in FIG. 4, some analyzer 21 of the plurality of analyzers 21 deciphers the psychoanalysis object information addressed to him or with no address by using the decryption software on his analyzer computer apparatus 20, and as shown in FIG. 5, he practices psychoanalysis of the pertinent client 2 on the basis of the acquired psychoanalysis object information by use of, for example, the collage therapy to prepare analysis result information.

In this case, the analyzer 21 performs psychoanalysis on the curriculum vitae of the client 2 and by reproducing the psychoanalysis object information by the analysis software on the screen. In reproducing the psychoanalysis object information, the procedure which has been taken for preparing the psychoanalysis object information is reproduced by the analysis software. To suit to the situation, the reproduction can be stopped, rapidly forwarded, and returned for progressing the analysis.

On the basis of where the above-mentioned photo A, the photo B, and the circle C are located, for example, the analyzer 21 concludes that the emotion of the client 2 is significantly unstable or that the emotion of the client 2 is normal (which is the technique as is used in the collage therapy).

The analysis result information thus prepared is ciphered by the encryption software, and is transferred to the client computer apparatus 1 as a file append to the electronic mail through the internet 30. In addition, the analysis result information is stored in the analyzer database 23.

In this case, if the analyzer 21 judges the mentality of the client 2 to be in the unstable or hazardous status before transferring the analysis result by means of the electronic mail, he takes such a step that a part of the analysis result information is temporarily stored in the client computer apparatus 1 as an immediate-opening-disabled mail (a mail which can be opened only when the specified period of time has elapsed from the time of the reception).

The client 2 uses the decryption software to decipher the psychoanalysis result information sent to his client computer apparatus 1 for knowing the psychoanalysis result for him. When a part of the psychoanalysis result information is sent as an immediate-opening-disabled mail, the client 2 can know the part after the specified time has elapsed.

Thus, the client 2 can visually know the psychoanalysis result information after the change of his mental state or after completion of the setup of his mind, with the mental oppression on him being alleviated. The above-mentioned psychoanalysis result information is stored in the client database 3.

According to the present remote psychoanalyzing system, the internet 30 can be used to transmit/receive a mail for taking the psychoanalysis procedure, and if the pertinent place is distant, remote psychological counseling can be performed.

If a clinical psychologist (qualified by such an authorization organization as the Japanese Certification Board for Clinical Psychologist, a private sector in Japan), who is the analyzer 21 to perform the psychological counseling, and the client 2, who is subjected to the psychological counseling, are remote from each other, practicing the psychoanalysis and transmission/reception of the psychoanalysis result information can be easily and rapidly performed, being not restricted by distance and time (time of day) (from any place and at any time).

Further, the client 2 to be subjected to the psychological counseling need not have a direct interview with the analyst 21 who is to perform psychological counseling, thus he can be lightheartedly subjected to the psychoanalysis. Other advantages include that the analyzer 21 can disclose the psychoanalysis result information in steps, that large-scale analysis can be performed with ease, and that a quantitative analysis can be performed.

Psychological counseling with the remote psychoanalysis system according to the present invention can be performed by using the collage therapy and other various types of therapy and means which have conventionally been known as those for psychological counseling.

According to the present invention as described above, the following effects will be provided.

First, psychoanalysis in a remote place (when the analyzer and the client are distant from each other) becomes possible, and preparing the psychoanalysis object information, making the analysis, and receiving the analysis result can be easily and rapidly performed, both the analyzer and the client being liberated from the restriction by distance and time.

Further, the procedure which has been taken for preparing the psychoanalysis object information can be reproduced in either the forward or backward direction; reversible psychological counseling and investigation can be performed; large-scale psychological counseling and analysis can be easily performed; and quantitative data of psychological counseling can be stored.

The psychoanalysis object information and the psychoanalysis result information are transmitted/received as an enciphered electronic mail, thus if the information should leaked to some other computer apparatus connected to the communication means, the privacy of the client and the analyzer can be protected by the encryption provided.

By sending the psychoanalysis result information as an immediate-opening-disabled mail, the metal pain of the client can be alleviated.

What is claimed is:

1. A remote psychoanalysis system using operation history having a client computer apparatus on the side of a client requesting psychoanalysis; a server computer apparatus on the side of a server controlling psychoanalysis information; a plurality of analyzer computer apparatuses on the side of analyzer practicing psychoanalysis; and communication means connecting said client computer apparatus, said server computer apparatus, and said plurality of analyzer computer apparatuses to one another through a network, comprising the steps of:
the client preparing psychoanalysis object information by using the client computer apparatus, and transmitting the psychoanalysis object information to the server computer apparatus through the communication means;
the server performing accounting for the client through said communication means for each matter of psychoanalysis request or confirmation of reception of money for the accounting;
the server transmitting said psychoanalysis object information prepared by the client from the server computer apparatus to the plurality of analyzer computer apparatuses through the communication means; and
some analyzer of the plurality of analyzers practicing psychoanalysis of the pertinent client on the basis of said psychoanalysis object information acquired by using his analyzer computer apparatus, and transmitting the psychoanalysis object information to the client computer apparatus through said communication means.

2. A remote psychoanalysis system using operation history as claimed in claim 1, wherein said psychoanalysis object information and said psychoanalysis result information are transmitted/received as a ciphered electronic mail.

3. A remote psychoanalysis system using operation history as claimed in claim 1 or 2, wherein, when said analyzer prepares analysis result information stating that the metal state of the client is unsteady, the analysis result information is transmitted to the client computer apparatus as an immediate-opening-disabled electronic mail, which cannot be opened before a specified date.
